(19) United States
(12) Patent Application Publication
(10) Pub. No.: US 2011/0092885 A1
(43) Pub. Date: Apr. 21, 2011

(54) Phacoemulsification Needle Tip With Bumps

(76) Inventor: Teruyuki Myoshi, Fukuyarna City (JP)

(21) Appl. No.: 12/533,351

(22) Filed: Jul. 31, 2009

Related U.S. Application Data
(60) Provisional application No. 61/092,357, filed on Aug. 27, 2008.

(51) Int. Cl. A61F 9/007

(52) U.S. Cl. 604/22

(57) ABSTRACT

A phacoemulsification needle has at least one grouping of surface protuberances formed on the tip inner surface, outer surface, or both. The protuberance groupings comprise three-dimensional protuberances of varying selected geometric shapes positioned proximate the lip of the phacoemulsification needle tip and provide surfaces to increase the efficacy of the phacoemulsification process.

### PRIORITY

This application claims priority from U.S. Patent Application 61/092,357, filed Aug. 27, 2008, which is incorporated herein in its entirety by reference.

### FIELD OF THE INVENTION

This disclosure relates to surgical instruments and surgical techniques used in eye surgery and more particularly, to designs for phacoemulsification needle tips for use with phacoemulsification handpieces.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. For example, it is now a common technique to take an artificial intraocular lens (IOL), fold it and insert the folded lens through the incision, allowing the lens to unfold when it is properly positioned within the capsular bag. Efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle at ultrasonic frequencies in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery and the prevention of such a collapse.

Phacoemulsification needles and tips are well represented in the prior art. Needles and tips of varying configurations are well known. A particular shape for a tip or needle is often dictated by the type of handpiece with which the needle is to be used.

U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published U.S. Patent Application No. 2003/0004455 (Kadziauskas) teaches and describes a bi-manual phaco needle using separate emulsification and aspiration needles inserted into the eye simultaneously during surgery.

Published U.S. Patent Application 2006/0217672 (Chon) teaches and describes a phacoemulsification tip having a crimped or swaged tip end.

U.S. Pat. No. 6,077,285 (Boukhny) teaches and describes a torsional ultrasound handpiece configured to impart both longitudinal and torsional motion to a phacoemulsification needle.

U.S. Pat. No. 6,402,769 (Boukhny) is a continuation in part of the '285 patent and further particularizes the frequencies at which the crystals providing both the torsional and longitudinal motion are activated.

U.S. Pat. No. 7,204,820 (Akahoshi) teaches and describes a phacoemulsification needle having an enlarged and rounded tip.

I have determined that forming a cluster of protuberances or “bumps” on the interior and/or exterior surface of a phacoemulsification needle tip will increase the efficiency and efficacy of the needle when used with both linear and torsional handpieces.

### BRIEF DESCRIPTION OF THE DRAWINGS

These and further aspects of the present invention will be best understood by reference to the accompanying drawings wherein:

FIG. 1 is a drawing showing prior art oval and square-shaped tips;

FIG. 2 is a drawing showing several prior art needle cross-sectional configurations;

FIG. 3 is a perspective view of a prior art phaco needle;

FIG. 4 is an enlarged detail of the tip of the needle of FIG. 3;

FIG. 5 is a right perspective view of a phacoemulsification needle tip embodying the present invention;

FIG. 6 is a right/lateral perspective view of the needle of FIG. 5;

FIG. 7 is a left perspective view of the needle of FIG. 5;

FIG. 8 is a lateral view of the needle of FIG. 5; and

FIG. 9 is an end view of a phacoemulsification tip illustrating the placement of bumps of different shapes

### DETAILED DESCRIPTION OF THE INVENTION

It is common for some in the art to refer to an entire phacoemulsification needle as a “tip”. For the purposes of the description that follows, the word “tip” shall refer to that portion of a phacoemulsification needle that is proximate the end of the needle that contacts the tissue to be emulsified. The portion of the needle extending from the tip to the needle mount shall be referred to as the “needle body”.

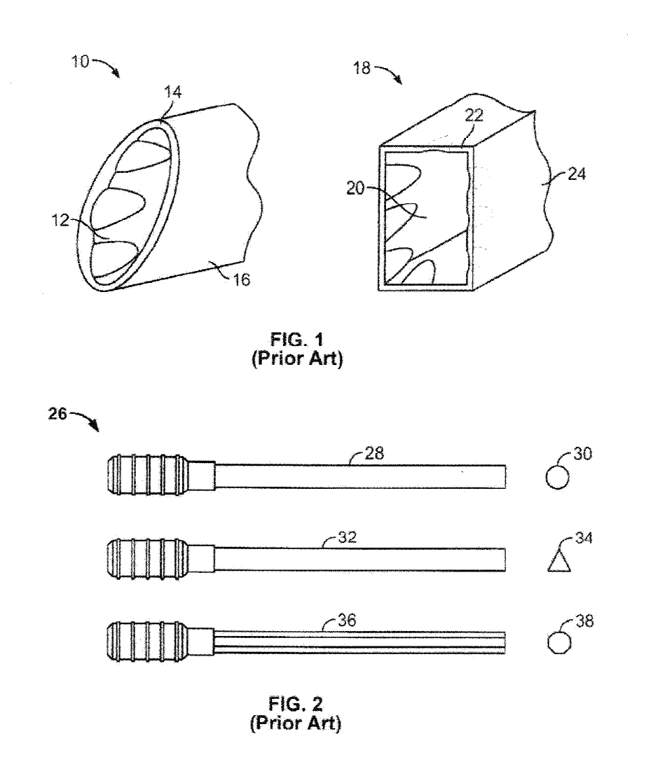

Referring now to FIG. 1, the numeral 10 indicates generally a prior art phacoemulsification needle tip as shown in U.S. Pat. No. 6,007,555. Needle 10 terminates in a mouth 12 defined by a lip 14 at the end of needle body 16, with lip 14 and needle body 16 formed as having an oval cross-section configuration.

Referring to FIG. 1, the numeral 18 indicates generally a prior art phacoemulsification needle tip from U.S. Pat. No. 6,007,555, having a mouth 20 defined by a lip 22 at the end of needle 24. The cross-sectional configuration of needle 18 and mouth 20 is a rectangle.

Referring now to FIG. 2, the numeral 26 identifies several prior art phacoemulsification needles as described in U.S. Pat. No. 5,725,495, with needle 28 having a body with a circular cross-section as shown at 30, needle 32 having a body with a triangular cross-section as shown at 34 and needle 36 having a body with an octagonal cross-section as shown at 38.

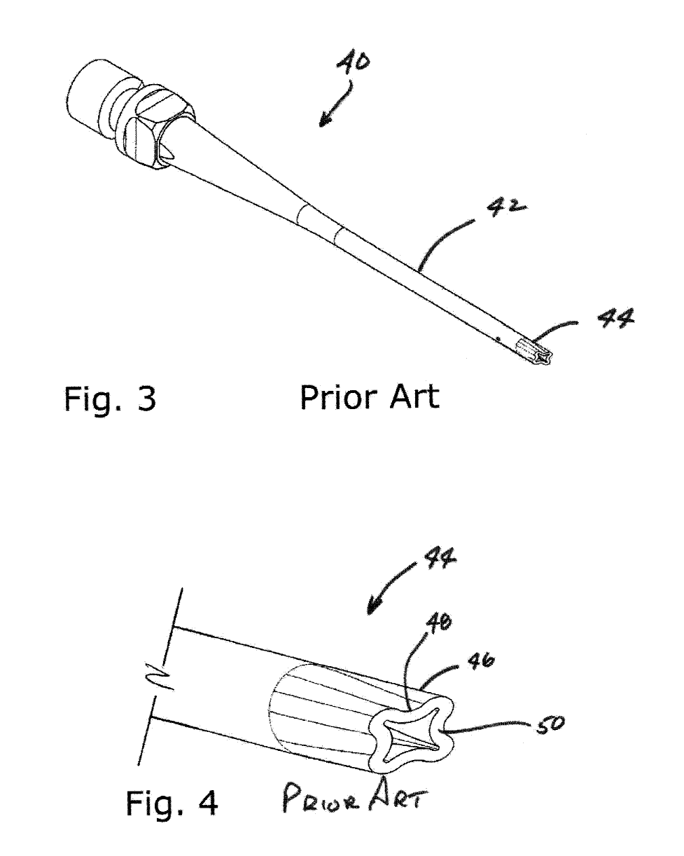

Referring now to FIG. 3 the numeral 40 indicates generally a prior art phacoemulsification needle as illustrated in Published U.S. Patent Application 2006/0217672 (Chon). Needle 40 has a shank 42 terminating in a tip 44.

Referring now to FIG. 4, tip 44 is shown in greater detail. As seen, and as described in the Chon publication, tip 44 is crimped or swaged to produce a plurality of ridges 46 and valley 48 resulting in the formation of a tip opening 50 of irregular shape. The resulting geometric configuration of tip 44 results in irregular surfaces such as ridges 46 and valleys 48 which are believed to make phacoemulsification more efficient. It can also be seen in FIG. 4 that the shape of tip opening 50 shows that the tip opening itself has been narrowed or made smaller.

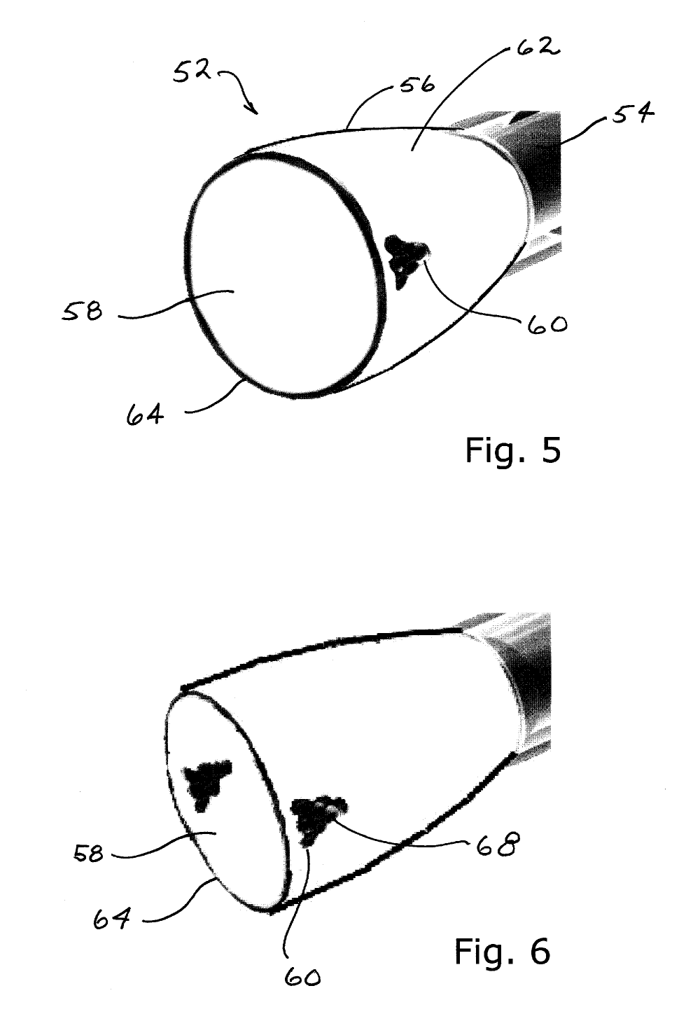

Referring now to FIG. 5, the numeral 52 identifies a phacoemulsification needle tip embodying principles of the present invention. For the purposes of this and the following descriptions, it is understood that tip 52 is formed at the end of needle shaft 54 which is of sufficient length to function effectively as a phacoemulsification needle assembly. It should also be understood that the principles of the present invention can be applied to phaco needles with straight configurations or angled configurations (such as the Alcon TurboSonics Flared ABS 30° Kellman Tip).

As seen in FIG. 5, tip 52 has a tip end 56 terminating at the tip opening 58. In the embodiment shown, tip opening 58 is circular but it should be understood that other shapes and configurations can be employed as well.

As seen in FIG. 5, a cluster 60 of protuberances or “bumps” are formed on the exterior surface 62 of tip end 56.

As seen in FIG. 6, cluster 60 is formed proximate lip 64 of tip opening 58. In the embodiment shown, bump cluster 60 is formed in a generally triangular pattern with the triangle base 66 formed proximate to and generally parallel to lip 64 and with the apex 68 of cluster 60 pointing generally rearward along tip end 56. It is contemplated that other configurations of bumps can be employed as well.

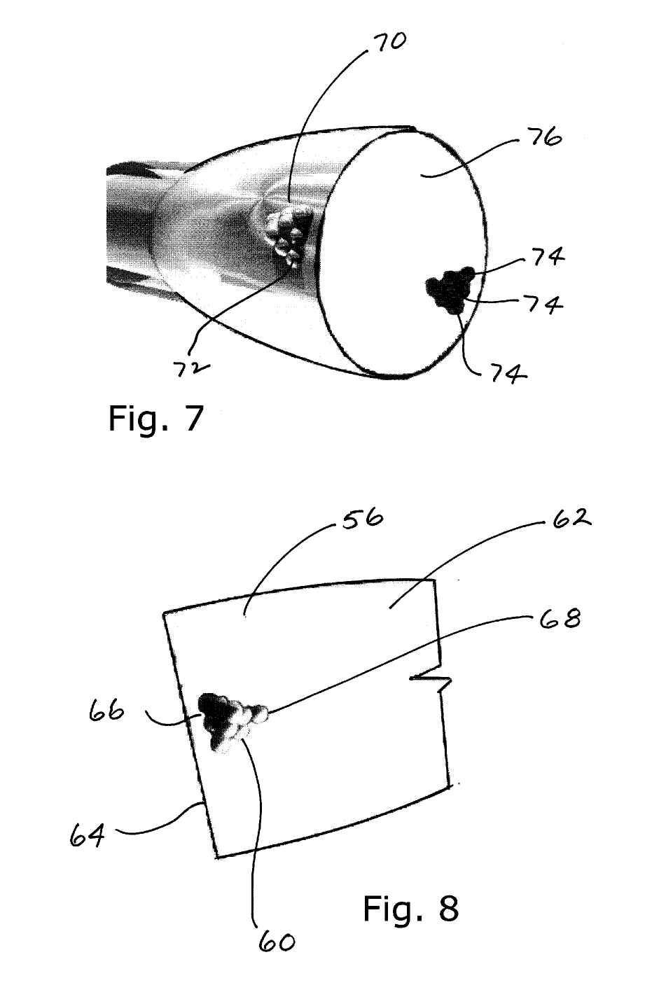

Referring now to FIG. 7, a second bump cluster 70 is shown. In this embodiment, cluster 70 is formed diametrically opposite to first cluster 60. As seen in greater detail in FIG. 7 and in FIG. 8, each individual bump 72 is shown as a generally hemispherical protuberance.

As seen in FIG. 7, first bump cluster 60 when viewed from within tip end 56 comprises a number of dimples 74. Dimples 74 are formed by pushing out inner surface 76 of tip end 56.

Referring to FIG. 8, first bump cluster 60 is shown more clearly showing base 66 parallel to and proximate to lip 64 of tip opening 58 and apex 68 extending rearward from tip opening 58. It can also be seen in FIG. 8 that the individual bumps in bump cluster 60 may protrude above outer surface 62 to varying heights as desired in a generally three-dimensional array. As seen in FIG. 7, one such array is generally pyramidal in shape.

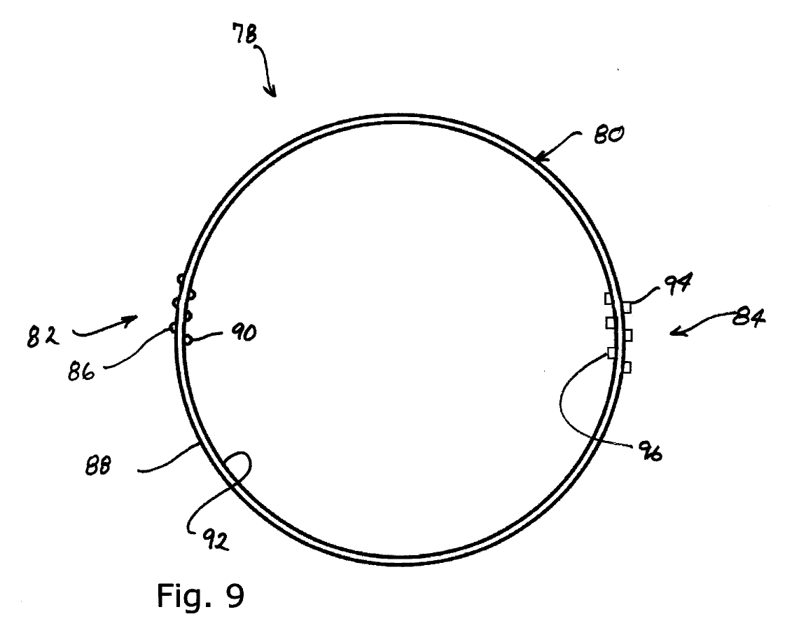

Referring now to FIG. 9 the numeral 78 identifies an end view of a phaco needle tip 80 upon which a pair of bump clusters 82, 84 are formed.

As described hereinabove, bump cluster 82 includes hemispherical outer bumps 86 extending outward from outer tip surface 88. Bump cluster 82 also includes hemispherical inner bumps 90 extending inwardly from inner surface 92.

Bump cluster 84 includes outer bumps 94 extending outward from outer surface 88 and inner bumps 96 extending inwardly from inner surface 92. Bumps 94 and 96 are square in shape. This illustrates that the bumps used in the present invention can be of various shapes and can extend from the inner surface or outer surface of the needle tip.

It should be understood that although the embodiment discussed herein shows a pair of diametrically opposed bump clusters, that other numbers and configurations of such clusters may be formed to alter the operating efficacy or operating characteristics of a phaco needle tip. It can also be seen that in this fashion, geometric protuberances are formed on such tips without closing off, narrowing or reshaping the tip opening.

While the foregoing describes an example or examples of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

What is claimed is:

1. A phacoemulsification needle for use with a handpiece, said needle comprising:
a needle body having a proximal end and a distal end,
said needle body having a central passageway;
a needle mount formed at said proximal end,
said needle mount adapted to attach said needle to said handpiece;
a needle tip formed at said distal end,
said needle tip having an inner surface and an outer surface;
said inner and outer surfaces terminating at a lip,
said lip defining a needle mouth communicating with said central passageway; and
at least one grouping of protuberances formed proximate said lip and protruding from said needle tip.

2. The apparatus as recited in claim 1 wherein said protuberances are formed on said inner tip surface proximate said lip.

3. The apparatus as recited in claim 2 wherein said protuberances are generally spherical in shape.

4. The apparatus as recited in claim 2 wherein said protuberances are generally cubical in shape.

5. The apparatus as recited in claim 2 wherein said grouping is formed in a generally triangular array of said protuberances.

6. The apparatus as recited in claim 2 wherein said protuberances protrude from said needle tip at varying heights from said inner surface.

7. The apparatus as recited in claim 6 wherein said protuberances form a generally three-dimensional array.

8. The apparatus as recited in claim 7 wherein said array is generally pyramidal.

9. The apparatus as recited in claim 2 wherein said tip comprises two said groupings positioned diametrically opposite one another.

10. The apparatus as recited in claim 2 wherein said tip comprises at least three said groupings,
said at least three groupings positioned equidistantly about said inner surface.

11. The apparatus as recited in claim 1 wherein said protuberances are formed on said outer tip surface proximate said lip.

12. The apparatus as recited in claim 11 wherein said protuberances are generally spherical in shape.

13. The apparatus as recited in claim 11 wherein said protuberances are generally cubical in shape.

14. The apparatus as recited in claim 11 wherein said grouping is formed in a generally triangular array of said protuberances.

15. The apparatus as recited in claim 11 wherein said protuberances protrude from said needle tip at varying heights from said inner surface.

16. The apparatus as recited in claim 15 wherein said protuberances form a generally three-dimensional array.

17. The apparatus as recited in claim 16 wherein said array is generally pyramidal.

18. The apparatus as recited in claim 11 wherein said tip comprises two said groupings positioned diametrically opposite one another.

19. The apparatus as recited in claim 11 wherein said tip comprises at least three said groupings,
said at least three groupings positioned equidistantly about said inner surface.

20. A phacoemulsification needle for use with a handpiece, said needle comprising:
a needle body having a proximal end and a distal end,
said needle body having a central passageway;
a needle mount formed at said proximal end,
said needle mount adapted to attach said needle to said handpiece;
a needle tip formed at said distal end,
said needle tip having an inner surface and an outer surface;
said inner and outer surfaces terminating at a lip,
said lip defining a needle mouth communicating with said central passageway;
at least one grouping of protuberances formed proximate said lip and protruding from said inner surface; and
at least one grouping of protuberances formed proximate said lip and protruding from said outer surface.

21. The apparatus as recited in claim 20 wherein at least some of said protuberances are generally spherical in shape.

22. The apparatus as recited in claim 20 wherein at least some of said protuberances are generally cubical in shape.

23. The apparatus as recited in claim 20 wherein at least one said grouping is formed in a generally triangular array of said protuberances.

24. The apparatus as recited in claim 20 wherein at least some said protuberances protrude from said needle tip at varying heights from said inner surface.

25. The apparatus as recited in claim 24 wherein said protuberances form a generally three-dimensional array.

26. The apparatus as recited in claim 25 wherein said array is generally pyramidal.

27. The apparatus as recited in claim 20 wherein said tip comprises two said groupings positioned diametrically opposite one another.

28. The apparatus as recited in claim 20 wherein said tip comprises at least three said groupings,
said at least three groupings positioned equidistantly about said inner surface.
